(19) United States
(12) Patent Application Publication
(10) Pub. No.: US 2006/0074386 A1
(43) Pub. Date: Apr. 6, 2006

(54) Device for producing medicinal foam

(75) Inventor: Jan-Christoph Wollmann, Bad Kreuznach (DE)

Correspondence Address: Paul D. Greeley, Esq.;Ohlandt, Greeley, Ruggiero & Perle, L.L.P., 10th Floor, One Landmark Square, Stamford, CT, 06901-2682, US

(73) Assignee: CHEMISCHE FABRIK KREUSSLER & CO. GmbH

(21) Appl. No.: 11/231,487

(22) Filed: Sep. 20, 2005

Related U.S. Application Data
(60) Provisional application No. 60/638,980, filed on Dec. 23, 2004.

(30) Foreign Application Priority Data
Oct. 5, 2004 (DE) 10 2004 048 749.9
Mar. 9, 2005 (DE) 10 2005 011 174.2

(51) Int. Cl. A61M 5/00

(52) U.S. Cl. 604/246

(57) ABSTRACT

A device for producing medicinal foam comprises a gas vessel for holding a sterile gas and an active agent vessel for holding an active agent. A connecting element connects both vessels. Further, a feeder for feeding the gas and the active agent back and forth between the two vessels is provided for producing the medicinal foam. The connecting element comprises a closure element for a sterile closure of one of the two vessels.

### BACKGROUND OF THE INVENTION

1. Field of the Invention

The invention refers to a device for producing in particular reproducible medicinal foam or bubble suspension of a gaseous and a liquid medium. In particular, the invention refers to a mixing device for a reproducible preparation and administration of injectables such as sclerosing agents, diagnostic agents, therapeutic agents, homeopathic agents and autologous blood, for example.

2. Related Prior Art

Sclerotherapy means the planned elimination of intracutaneous, subcutaneous and/or transfascial varices and the sclerotization of subfascial vessels in case of venous anomalies by injecting a sclerosing agent. The various sclerosing agents cause damage to the endothelium of the vessels. Thereafter, a secondary vascular occlusion occurs and, in the long term, the veins are transformed into a strand of fibrous tissue, i.e. sclerosis occurs. It is the purpose of the sclerotization treatment to definitely transform the veins into a fibrous strand. This can not recanalize and, in its functional result, corresponds to the surgical procedure for removing a varice. Besides a sclerotization with liquid sclerosing agents, the sclerotization with foamed sclerosing agents becomes ever more important. The foam remains in vein for a longer period. Here, surfactant sclerosing agents, such as Polidocanol, are most often made to achieve a foamy state by pumping the agent back and forth between two pumps or by shaking, whereafter it is injected in a conventional manner. At present, there is no approved technique that would allow a reproducible preparation of a standardized foam.

Further, a plurality of preparations suited for use as ultrasonic contrast media are known, some of which contain surfactants that support the formation of bubbles and stabilize these. The bubbles or a foam reflecting ultrasound are the true contrast medium and are produced only immediately before being administered.

A mixing device for producing medicinal foam or for producing bubbles is known from EP 0 564 505. Here, a mixer with a helically shaped mixing element is described. The mixer is an accessory element that may be permanently connected to a syringe. When a liquid and/or gaseous medium is expelled from a second syringe, the medium reaches the mixer that contains the gas in a defined volume and nature. Here, the gaseous phase and the liquid phase are mixed along the helical mixing element. Thereby, a therapeutic and/or diagnostic foam may be produced. Due to the helical mixing elements arranged in the mixer, the mixer is a component that can only be produced as an injection molded part with intricate injection molds.

Especially in producing foams for medicinal use, especially for sclerotherapy, it is necessary to produce a sterile foam. Should air be used to produce foam, it is possible to aspire air through a sterile filter into a syringe and to use the sterile air thus obtained to produce foam. However, this has the drawback of requiring an additional step and an additional component in the form of the sterile filter. This increases the costs. Further, the volume of waste is augmented.

It is further known from GB 2 369 996 to close a syringe filled with sterile air using a three-way valve. A second valve is filled with an active agent and is also connected to the three-way valve so that the syringes are oriented under an angle of 90° to each other. Thereafter, the three-way valve is rotated to a position in which both syringes are in communication so that by pumping the gas and the active agent back and forth, foam can be produced. Closing a syringe filled with sterile air using a three-way valve has the drawback that, for example, during transport or handling, inadvertent opening and thus a contamination and/or a change in the gas volume can occur. Furthermore, the handling of this system is complex, since after both syringes have been connected to the three-way valve, the latter also has to be opened. Moreover, it is difficult to produce a reproducible foam with this device, since the diameter of the passage changes already at a slightly false position of the three-way valve. This can cause the production of a foam with a different size of bubbles. Further, the orientation of the two syringes under an angle of 90° is disadvantageous, since this makes the handling more difficult.

Typically, after the production of foam both syringes contain foam. With the device described in GB 2 369 996, for therapy, one of the syringes filled with foam has to be unscrewed from the three-way valve. In order to additionally inject the foam remaining in the second syringe at a later time, if necessary, it is required to close the three-way-valve so as to avoid contamination. To then inject the remaining foam, the first syringe just used for therapy must be screwed to the three-way valve again and the valve has to be opened, to then transfer the remaining foam into the injection syringe, for example. Thus, this procedure is extremely complex.

It is an object of the invention to provide a device for producing medicinal foam from a gaseous and a liquid medium, the device being adapted to meet high sterility demands.

### SUMMARY OF THE INVENTION

The present device for producing medicinal foam that is particularly suitable for use in scleroscopy, comprises a gas vessel for holding a sterile gas, especially sterile air. Further, an active agent vessel for holding a usually liquid active agent is provided. Preferably, both vessels are syringes, in particular disposable syringes. Both vessels are adapted for fluidic connection to a connecting element. Moreover, a feed means is provided for conveying the gas and the active agent back and forth between both vessels so as to produce the medicinal foam. In a preferred embodiment, the feed means comprises two feed elements, each feed element being connected with one of the two vessels, respectively. Preferably, the feed elements are the syringe pistons.

According to the invention, the connecting element is connected with one of the vessels, preferably the gas vessel. The connection is obtained for example by screwing, especially by means of a Luer lock. Similarly, the connecting element may be permanently connected with the vessel, e.g. glued thereto or formed integrally therewith. Specifically, the hub with the opening of the syringe can be formed as a connecting element. Preferably, the connecting element is surrounded by the Luer lock. According to the invention, the connecting element comprises a closure element for the sterile closing of the vessel. Thus, it is possible to provide a sterile gas, e.g. sterile air, in one of the two vessels, especially in the gas vessel, which can not escape from the vessel because of the closure element provided. An undesired intrusion of non-sterile air is also avoided because of the closure element provided according to the invention. Thus, in a particularly preferred embodiment, the connecting element is configured such that in the unconnected state, i.e. especially before the gas vessel together with the connecting element is connected to the active agent vessel, both an intrusion and an escape of gas and/or liquid into or from the vessel closed by the closure element is prevented. This has the advantage of ensuring a very good sterility of the medium contained in the vessel. Further, it is ensured that an unintentional change of the gas volume is avoided. Thereby, a good reproducibility of the medical foam is ensured.

Preferably, the closure element comprises a resilient rubber stopper. The stopper may comprise a slit serving to open the closure element. The slit is configured such that the slit walls abut each other in the unconnected state and close the vessel tightly, such that an intrusion or an escape of gas and/or liquid is avoided.

Preferably, the closure element is opened automatically upon connecting the connecting element with the second vessel, in particular the second syringe. In a preferred embodiment, this guarantees that by opening in the manner provided by the invention contamination is avoided by the connection, other than when removing a closure element in the form of a lid or the like. Further, no additional step such as removing a lid or opening a valve is required. According to the invention, this specifically allows to provide a closure element with a significantly lower risk of contamination.

The present device for producing medicinal foam has the particular advantage that the sterile gas is preferably already present in a sterile condition and does not have to be obtained first through a sterile filter. Further, due to the automatic opening, the gas remains sterile so that an inadvertent contamination by aspiration is avoided. Moreover, it is guaranteed that the exactly defined volume of gas, and thus the mixing ratio of gas and agent, will not be corrupted for example by an unintentional escape of gas. Thus, it is possible to produce a reproducible foam and to create a standard.

Creating a high standard or a high uniformity of the foam producible with the present device can be improved further by also prefilling the second vessel. To this effect, the second vessel, which is especially the active agent vessel, is closed by a closure member. The closure member may be configured similar to the closure element, in particular as a membrane or a plastics stopper. When two prefilled vessels are provided, one of which is closed with the connecting element including the closure element, this is further advantageous in that a further process step, i.e. filling the still empty vessel, usually the active agent vessel, can be omitted.

Preferably, the closure element is opened by penetrating a membrane of the closure element. The penetration of the membrane may be effected by a hub on the second vessel, especially provided at the syringe, in particular the hub of a Luer lock. In particular, the closure element is opened such that the process is reversible and the closure element therefore closes the vessel again, when in the unconnected state. Specifically, the membrane or the plastics stopper are configured such that it has a slit which may be spread open by means of a tubular element, for example, and closes again when the tubular element is withdrawn.

In a particularly preferred embodiment, the connecting element comprises a tube element. When connecting the second vessel with the connecting element, the membrane is penetrated by the tube. For this purpose, the closure element and/or the tube element are preferably arranged for displacement in the connecting element. Here, the tube element is preferably held fixed in the connecting element so that a displacement of the closure element causes a penetration of the membrane by the tube element. The closure element is preferably displaced by a hub of the vessel, in particular the Luer lock hub of a syringe.

As soon as the vessels are connected through the connecting element, the gas and the active agent can be pumped back and forth between the two vessels, in particular the two syringes, to produce the sterile foam. In doing so, the gas and the active agent preferably flow through the tube element. Specifically, there is no flowing around the closure element. This is advantageous in that clearly defined flow paths and thus a clearly predeterminable flow behaviour are given. This increases the reproducibility of the medicinal foam.

To ensure a secure closing of the first vessel prior to the connection with the second vessel, the closure element is preferably spring-loaded. Upon opening the closure element, the closure element is preferably urged against the spring force. The spring force may be caused, for example, by a coil spring or another resilient element. The provision of such a closure element guarantees that the filling amount in the vessel remains constant and is not changed, e.g., during transport or handling. Further, the gas vessel can be closed again and has good sterility.

Preferably, the connecting element comprises a mixing element. It is particularly preferred that the tube serving to open the membrane is designed as a mixing element. It is sufficient to provide a tubule with a small cross section so that turbulences are generated by the change in cross section, which turbulences serve to intermix the active agent with the gas. The tube element, preferably made of stainless steel or having a coating resistant against the active agent, has an inner diameter of up to 3 mm, for example. One of the openings of the tube may have its cross section reduced directly or indirectly by providing an additional element. The cross section is preferably reduced to a cross section of 0.3-2 mm. Tests have shown that a medicinal foam of very high quality can thereby be obtained with a very good reproducibility. In addition, barriers, deflecting elements and the like may for example be provided within the mixing element to ensure the generation of sufficient turbulences.

The present device has the particular advantage that, due to the design of the connecting element, it is not necessary, for example after the production of a foam, to close a valve or the like, to avoid contamination of foam remaining in one of the syringes, for example. This is not necessary since an automatic closure is performed by the closure element when the syringe is removed from the connecting element. In particular for a later removal of the foam remaining in the syringe, a simple and safe reconnection of the syringe used for injection and the connecting element can be made. The handling of the present device is thus very simple while ensuring great safety.

Further, the invention refers to a vessel, such as a syringe, particularly useful in the present device. The vessel, preferably filled with gas, is connected to a connecting element comprising a closure element. The connecting element, particularly adapted to be connected with a second vessel, especially a second syringe, is preferably embodied as described above.

The invention further refers to a kit for producing medicinal foam comprising the above described first vessel, in particular filled with gas and closed with the connecting element. Moreover, the kit comprises a second vessel which, as the first vessel, is a syringe, in particular. In addition, the kit may comprise an active agent vessel, such as an active agent ampoule containing a sclerosing agent, for example. To produce the medicinal foam, the active agent is filled from the active agent vessel into the second vessel. Preferably, this is done by suction into the second vessel configured as a syringe. Possible, the kit my additionally comprise a needle for that purpose.

In an alternative embodiment of the kit, the second vessel, in particular the second syringe, instead of the active agent vessel is already filled with an active agent and closed with a closure means as described above.

In a particularly preferred embodiment of the kit the two vessels, which are conventional syringes in particular, are prefilled and connected with each other through the connecting element. However, the connection is such that the closure element of the connecting element is not yet open. This may be achieved, for example, by the fact that the second vessel, especially the second syringe, is not yet fully screwed to the connecting element using the Luer lock. The connection between the two vessels is then established by fully screwing or connecting the second vessel with the connecting element.

Such a kit has the particular advantage that the medicinal foam can be produced very quickly. Not tome consuming preparatory steps are required. This may increase acceptance with practitioners. Further, the risk of contamination while connecting individual components is avoided.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying drawings in which

In the figures:

FIG. 1 is a schematic sectional side view of the connecting element,

FIG. 2 is a schematic sectional side view of the connecting element connected with two vessels,

FIG. 3 is a schematic sectional partial view of the connecting element together with the closure element of another embodiment,

FIG. 4 a schematic top plan view of the embodiment illustrated in FIG. 3, and

FIGS. 5-7 schematic sectional partial views of the connecting element together with the closure element of other embodiments.

### DESCRIPTION OF PREFERRED EMBODIMENTS

A connecting element 10 comprises a cylindrical hub 12 with an inner thread 14. A channel 18 is formed within the hub 12 by a tube element 16, especially of circular cross section. A tube 20 is arranged in the channel 18, extending over almost the entire length of the connecting element. At one end face 22, the tube element 16 has an opening 24 opening into the channel 18.

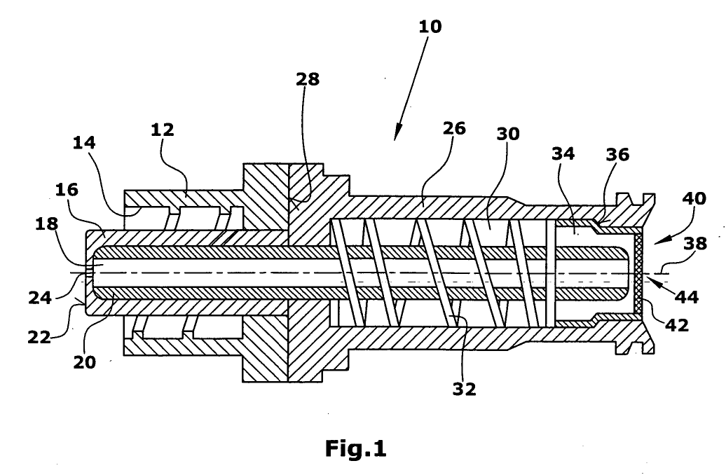

A housing element 26 is connected with the hub 12. The connection may be obtained along a contact surface 28 by glueing. Similarly, the two parts may be screwed together or connected otherwise. A circular cylindrical cavity 35 is formed in the housing 26. A coil spring 32 is arranged in the cavity 30, which urges a closure element 34, also provided in the cavity 30, outward against a stop 36 which, in the embodiment illustrated, is a chamfer. The closure element 34, which comprises a membrane 42 and a sleeve in the embodiment illustrated, is a resiliently deformable element which can be pushed into the housing element of FIG. 1 from the right in a compressed condition, restores to its original shape within the housing 26 and is then held in the housing 26 because of the stop 36. Similarly, it is possible to design the housing 26 as two parts to facilitate the mounting of the closure element 34. in this instance, the housing 26 can be separated such that the closure element 34 is possible from the left in FIG. 7.

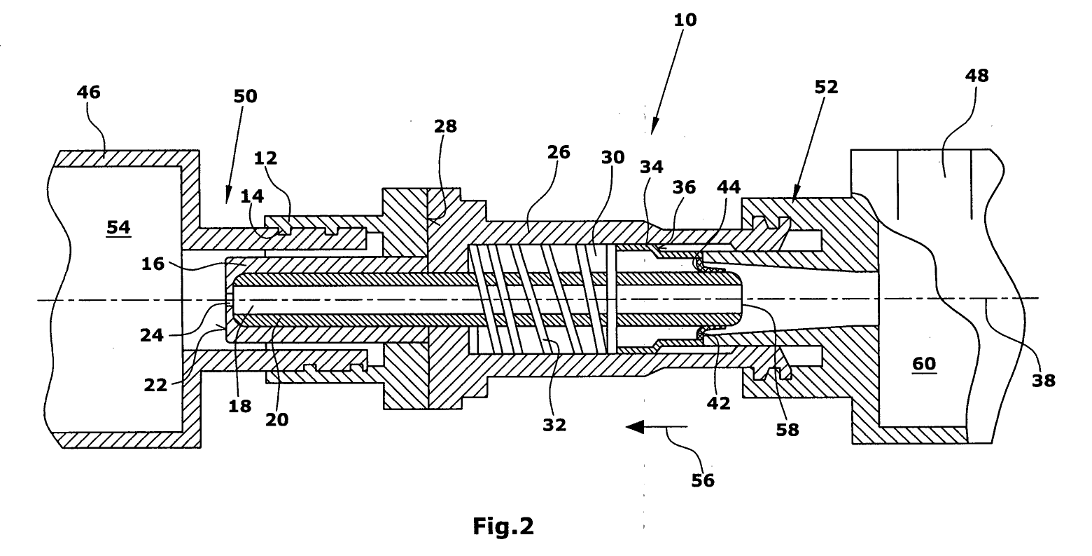

The closure element, as well as the housing 26 and the hub 12, is rotational symmetric to a center line 38 of the closure element 10. The front side 10 of the closure element 34 is closed by a membrane 42. The membrane 42 has a slit 44. The slit 44 is illustrated in the drawing for the sakes of clarification. Actually, the membrane portions abut each other in the state illustrated in FIG. 1 so that the slit 44 is closed, yet may be opened easily (FIG. 2).

The connecting element 10 may be connected with a gas vessel 46 and an active agent vessel 48, the two vessels 46, 48 preferably being conventional syringes with Luer lock connections 50 or 52, respectively. For transport and prior to mixing the gas in the gas vessel 46 with the active agent present in the active agent vessel 48, only the gas vessel 46 is connected to the connecting element. To do this, the Luer lock connector 50 of the gas vessel 46 is screwed into the hub 12. Because of the opening 24, a fluidic connection exists between the inner space 54 of the gas vessel 46 and the channel 18 in which the tube 20 is arranged.

Prior to inserting or screwing the liquid vessel 48 or the Luer lock 52, respectively, the vessel 46 is closed tightly due to the closure element 34.

By screwing or inserting the Luer lock 52 into the housing 26, the closure element 34 is pushed into the connecting element 10 in the direction of the arrow 56. Here, the slit 44 of the membrane 42 is opened or the membrane 42 is penetrated. Because of the opening 58 provided in the tube, the inner space 60 of the active agent vessel 48 is also fluidically connected with the channel 18.

By actuating the syringe pistons or a feed means, the active agent may be pumped from the inner space 60 through the tube 20 or the channel 18 into the inner space 54, and the gas may be pumped from the inner space 54 through the tube 20 into the inner space 60. This causes an intermixing of the gas and the active agent and then the gas and the active agent are pumped back and forth together between the two spaces 54, 60. Thus, the medicinal foam is produced. This has the advantage that the force exerted for example on the syringe piston, as well as the pump rate can be adjusted or are defined. Thereby, the standardization of the foam produced is further enhanced.

The tube 20 serves as the mixing element and may possibly comprise additional deflecting or mixing elements inside. Further, deflecting or mixing elements can also or additionally be arranged at the inlet and/or the outlet of the tube 20. Possibly, in addition to or instead of the above described mixing elements, mixing elements may also be provided in other regions of the devices through which the active agent and the gas flow. Moreover, the length off the pipe 20 is selected feasibly, in particular empirically. According to the invention, the change in cross section caused by the opening 24 and the opening 58 is sufficient for intermixing.

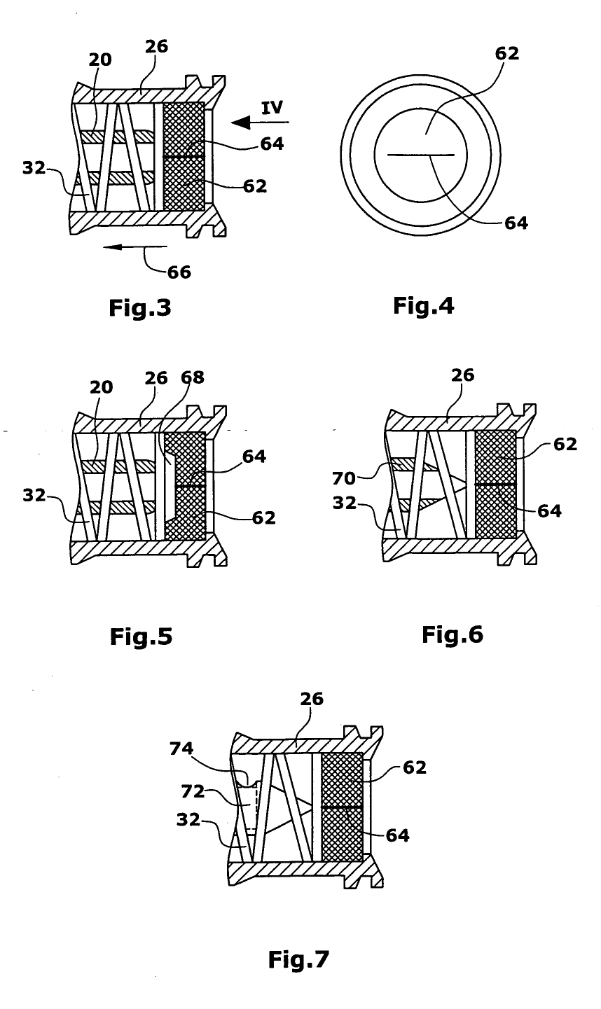

FIGS. 3 to 7 illustrate further embodiments of the present connecting element with different closure elements. In FIGS. 3-7, identical or similar components are given the same reference numerals.

In the embodiment shown in FIGS. 3 and 4, a plastics or rubber stopper 62 is provided as a closure element in the housing 26. The plastics stopper 62 may be mounted as described above with reference to the closure element 34. the plastics stopper 62 has a slit 64 which is spread apart when the plastics stopper 62 is moved in the direction of the arrow 66. When the plastics stopper 62 is pushed back into the position shown in FIG. 3 by the spring 32, the slit 64 is closed again automatically.

In the embodiment illustrated in FIG. 5, the plastics stopper 62 additionally comprises a recess 68 directed towards the tube 20, so that the tube 20 is positively guided and a secure opening of the slit is guaranteed.

In the embodiments shown in FIGS. 6 and 7, the plastics stopper 62 is not opened with the aid of the tube 20, but with a needle 70 or 72, respectively. In this case, the needle 70 is either open in the direction of the stopper or the needle 72 has a lateral opening 74. By providing the lateral opening 74 turbulences are created which, depending on the active agent used, allow for an enhanced production of foam. When needles 70, 72 are provided, the slits 64 may be omitted.

Although the invention has been described and explained with reference to specific illustrative embodiments thereof, it is not intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

What is claimed is:

1. A device for producing medicinal foam, comprising
a gas vessel for holding a sterile gas,
an active agent vessel for holding an active agent,
a connecting element for connecting the gas vessel with the active agent vessel, and
a feed means for feeding the gas and-the active-agent back and forth between the two vessels to produce the medicinal foam,
wherein said connecting element is connected with one of the vessels and comprises a closure element for a sterile closure of the vessel.

2. The device of claim 1, wherein the closure element may be opened by connecting the connecting element with the second vessel.

3. The device of claim 2, wherein the opening is effected by penetrating a membrane provided in particular at the closure element.

4. The device of claim 3, wherein the connecting element comprises a tube which penetrates the membrane when the two vessels are connected.

5. The device of claim 1, wherein the closure element is displaceable within the connecting element.

6. The device of claim 1, wherein the closure element is spring-loaded.

7. The device of claim 1, wherein the connecting element comprises a mixing element.

8. The device of claim 7, wherein the mixing element is the tube provided in the connecting element.

9. The device of claim 1, wherein the feed means comprises one feed element per vessel.

10. The device of claim 1, wherein the closure element is configured such that in the unconnected state both an intrusion and an escape of gas and/or liquid into or from the vessel is prevented.

11. The device of claim 1, wherein the closure element comprises a resilient plastics stopper.

12. The device of claim 1, wherein the closure element comprises a slit for being opened.

13. The device of claim 4, wherein, in the connected state, the active agent and the gas flow through the tube element.

14. The device of claim 1, wherein the first vessel, closed by the closure element of the connecting element, is prefilled.

15. The device of claim 1, wherein the second vessel is closed by a closure member and is prefilled, the closure member preferably being configured corresponding to the closure element.

16. A vessel for a device which comprises: a gas vessel for holding a sterile gas; an active agent vessel for holding an active agent; a connecting element for connecting the gas vessel with the active agent vessel; and a feed means for feeding the gas and the active agent back and forth between the two vessels to produce the medicinal foam, wherein said connecting element is connected with one of the vessels and comprises a closure element for a sterile closure of the vessel, said vessel being filled with gas and closed by a connecting element, said connecting element comprising a closure element for sterile closure.

17. A kit for producing medicinal foam. Comprising
a first vessel for a device which comprises: a gas vessel for holding a sterile gas; an active agent vessel for holding an active agent; a connecting element for connecting the gas vessel with the active agent vessel; and a feed means for feeding the gas and the active agent back and forth between the two vessels to produce the medicinal foam, wherein said connecting element is connected with one of the vessels and comprises a closure element for a sterile closure of the vessel, said vessel being filled with gas and closed by a connecting element, said connecting element comprising a closure element for sterile closure, and
a second vessel.

18. The kit of claim 17, wherein an active agent vessel is additionally provided containing an active agent for the second vessel.

19. The kit of claim 17, wherein the second vessel is prefilled with active agent and closed.

20. The kit of claim 19, wherein the first vessel is connected with the second vessel through the connecting element, whereas the closure element is still unopened.
